(19) United States
(12) Patent Application Publication
(10) Pub. No.: US 2005/0153347 A1
(43) Pub. Date: Jul. 14, 2005

(54) Analysis of methylation status using oligonucleotide arrays

(75) Inventors: Michael H. Shapero, Redwood City, CA (US); Guoying Liu, Emeryville, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 11/020,528

(22) Filed: Dec. 23, 2004

Related U.S. Application Data
(63) Continuation of application No. 10/841,027, filed on May 7, 2004.
(60) Provisional application No. 60/468,925, filed on May 7, 2003.

(51) Int. Cl. C12Q 1/68; C12P 19/34

(52) U.S. Cl. 435/6; 435/91.2

(57) ABSTRACT

The present invention provides for novel methods and kits for determining the methylation status of a cytosine in a nucleic acid sample. The methylation status of a plurality of cytosines may be determined simultaneously. In one embodiment methylation status is determined using methylation specific modification of cytosines followed by locus specific amplification, single base extension at the interrogation position and identification of the extended base by array hybridization. In another embodiment methylation specific modification of a cytosine is detected by hybridization to an array of probes that are perfectly complementary to either the methylated product of modification or the unmethylated product of modification. In another embodiment methylation status is determined using methylation specific restriction enzymes coupled with hybridization to an array.

### RELATED APPLICTIONS

The present application claims priority to U.S. Provisional Application No. 60/468,925, filed May 7, 2003 the disclosure of which is incorporated herein by reference in its entirety.

### FIELD OF THE INVENTION

The invention relates to analyzing the methylation status of selected cytosine residues using arrays. In some embodiments target sequences are subjected to a methylation sensitive treatment. In some embodiments the methylation sensitive treatment is sodium bisulfite treatment. In some embodiments the methylation sensitive treatment is digestion with restriction enzymes that recognize the same restriction site but are differentially sensitive to methylation. In some embodiments the invention relates to the preparation of target for array based analysis of methylation. The present invention relates to the fields of molecular biology and genetics.

### BACKGROUND OF THE INVENTION

The genomes of higher eukaryotes contain the modified nucleoside 5-methyl cytosine (5-meC). This modification is usually found as part of the dinucleotide CpG. The frequency of this dinucleotide is under represented in the human genome, and CpG islands are often located near the 5′ end of transcribed sequences. Patterns of CpG methylation are heritable, tissue specific, and correlate with gene expression. Transcriptionally inactive genes contain 5-meC whereas transcriptionally active genes do not. Thus the identification of sites in the genome containing 5-meC is important in understanding cell-type specific programs of gene expression and how gene expression profiles are altered during both normal development and diseases such as cancer. Precise mapping of DNA methylation patterns in CpG islands has become essential for understanding diverse biological processes such as the regulation of imprinted genes, X chromosome inactivation, and tumor suppressor gene silencing in human cancer.

### SUMMARY OF THE INVENTION

In one embodiment a method is provided for determining if a cytosine in a target sequence in a nucleic acid sample is methylated. A nucleic acid sample is fragmented by, for example, digestion with a restriction enzyme and an adaptor with a common priming sequence is ligated to the fragments. The nucleic acid sample is modified so that methylated and unmethylated cytosines are differentially modified. This may be done by, for example, sodium bisulfite modification which changes unmethylated cytosines to uracil but leaves methylated cytosines unchanged. The presence or absence of modification is detected using an array of oligonucleotide probes.

In one embodiment at least one capture probe is hybridized to the modified sample. A capture probe may be complementary to a region immediately upstream of the cytosine to be interrogated for methylation. The capture probe may be extended by a single base complementary to the base at the position of the cytosine being interrogated. The identity of the incorporated base may be determined using an array of tag probes that are complementary to tag sequences in the capture probe. The tag probes may be attached to a solid support that is for example a planar support or beads.

In another embodiment capture probes comprises a second common priming sequence, a tag sequence, a recognition sequence for a type IIS restriction enzyme and a region that is complementary to a target sequence. In some embodiments capture probes are designed for each cytosine to be interrogated. Capture probes hybridize to the target sequence 3′ of the cytosine so that they may be extended through the position of the cytosine. The type IIS recognition sequence is positioned so that cleavage will occur between the position of the cytosine being interrogated and the base that is immediately 5′ to that position. Capture probes are extended and amplified. The amplified fragments are digested with the Type IIS restriction enzyme and the fragments are extended in the presence of at least one labeled ddNTP so that a single ddNTP corresponding to the position of the cytosine being interrogated is incorporated. The extended products are hybridized to an array to detect the ddNTPs that are incorporated. In many embodiments the array is an array of probes that are complementary to the tag sequences in the capture probes. The methylation status of the cytosine is determined from the identity of labeled ddNTPs incorporated. The label may be, for example, biotin or chemiluminescent.

In some embodiments the ddNTPs used are ddGTP and ddATP which may be incorporated in separate reactions that may be hybridized to separate arrays. In some embodiments ddCTP and ddTTP are also used. When sodium bisulfite modification is used incorporation of ddGTP indicates the cytosine is methylated and ddATP indicates the cytosine is unmethylated. If two copies of the gene containing the cytosine of interest are present one may be methylated while the other is unmethylated so both ddATP and ddGTP would be incorporated. If the gene is present in more than two copies a ratio of unmethylated to methylated may be determined.

In some embodiments the fragmented nucleic acid sample is not ligated to an adaptor. The extended capture probes are made double stranded by hybridizing target specific reverse primers to the extended capture probes. The target specific reverse primers comprise a generic priming site so the double stranded capture probes are then amplified with generic primers. In this embodiment a Type IIS recognition site can be introduced in either the capture probe or the target specific reverse primer.

In some embodiments the methylation status of a cytosine of interest is determined in a plurality of individuals. Methylation status may be correlated with disease status. In some embodiments the methylation status of a plurality of cytosines of interest are determined from a plurality of individuals.

In some embodiments kits for the determination of methylation status of one or more cytosines are provided.

In another embodiment a method for determining if a cytosine of interest is methylated using methylation specific restriction digestion and an array of probes is provided.

### BRIEF DESCRIPTION OF THE FIGURES

FIG. 1A shows a schematic for a method to determine methylation status of a cytosine using methylation specific modification and a tag array.

FIG. 1B shows a schematic of the modification step, the extension step and the amplification step of one embodiment.

FIG. 1C shows a schematic of the Type IIS restriction enzyme cleavage step, the mini sequencing step and the array hybridization step of one embodiment.

FIG. 2 shows a schematic for a method to determine methylation status of a cytosine using methylation specific modification and a tag array using two target specific primers.

FIG. 3A shows a schematic for a method to determine methylation status of a cytosine using methylation specific restriction digestion and a target specific array.

FIG. 3B shows a schematic of the possible outcomes expected when a genotyping array, for example the Mapping 10K or 100K Arrays, is used to detect fragments in combination with whole genome sampling assays (WGSA).

FIG. 4A shows a schematic for a method to determine methylation status of a plurality of cytosines using sodium bisulfite modification, amplification of a subset of fragments using WGSA.

FIG. 4B shows detection of the WGSA amplification product by hybridization to an array of target specific probes that has probe sets that hybridize specifically to either the methylated target which has a C:G base pair after modification or the unmethylated target which has an A:T base pair after modification.

FIG. 4C shows an example of design of a probe set to detect sites of methylation after treatment of DNA with sodium bisulfite. Unmethylated and methylated sites are detected as though the position was a SNP with alleles T or C.

### DETAILED DESCRIPTION OF THE PREFERRED EMBODIMENTS

(A.) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89: 117 (1990)), transcription amplification (Kwoh et al., Proc. Natl. Acad. Sci. USA 86, 1173 (1989) and WO88/10315), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245), self-sustained sequence replication (Guatelli et al., Proc. Nat. Acad. Sci. USA, 87, 1874 (1990) and WO90/06995) and nucleic acid based sequence amplification (NABSA). (See, U.S. Pat. Nos. 5,409,818, 5,554,517, and 6,063,603, each of which is incorporated herein by reference). The latter two amplification methods involve isothermal reactions based on isothermal transcription, which produce both single stranded RNA (ssRNA) and double stranded DNA (dsDNA) as the amplification products in a ratio of about 30 or 100 to 1, respectively. Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (3^{rd} Ed. Cold Spring Harbor, N.Y, 2002); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170 and U.S. Patent Pub. Nos. 20040024537, 20040002819, 20040002818 and 20040002817.

Additionally, the present invention may have preferred embodiments that include methods for providing genetic information over networks such as the Internet as shown in U.S. patent application Ser. Nos. 10/063,559, 60/349,546, 60/376,003, 60/394,574, 60/403,381.

(B.) Definitions

Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferably at least 8, 15 or 20 nucleotides in length, but may be up to 50, 100, 1000, or 5000 nucleotides long or a compound that specifically hybridizes to a polynucleotide. Polynucleotides of the present invention include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “fragment,” “segment,” or “DNA segment” refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations (see, for example, U.S. Ser. No. 09/358,664). Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3^{rd} Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful.

A number of methods disclosed herein require the use of restriction enzymes to fragment the nucleic acid sample. In general, a restriction enzyme recognizes a specific nucleotide sequence of four to eight nucleotides and cuts the DNA at a site within or a specific distance from the recognition sequence. For example, the restriction enzyme EcoRI recognizes the sequence GAATTC and will cut a DNA molecule between the G and the first A. The length of the recognition sequence is roughly proportional to the frequency of occurrence of the site in the genome. A simplistic theoretical estimate is that a six base pair recognition sequence will occur once in every 4096 (4⁶) base pairs while a four base pair recognition sequence will occur once every 256 (4⁴) base pairs. In silico digestions of sequences from the Human Genome Project show that the actual occurrences may be more or less frequent, depending on the sequence of the restriction site. Because the restriction sites are rare, the appearance of shorter restriction fragments, for example those less than 1000 base pairs, is much less frequent than the appearance of longer fragments. Many different restriction enzymes are known and appropriate restriction enzymes can be selected for a desired result. (For a description of many restriction enzymes see, New England BioLabs Catalog which is herein incorporated by reference in its entirety for all purposes).

Type-IIs endonucleases are a class of endonuclease that, like other endonucleases, recognize specific sequences of nucleotide base pairs within a double stranded polynucleotide sequence. Upon recognizing that sequence, the endonuclease will cleave the polynucleotide sequence, generally leaving an overhang of one strand of the sequence, or “sticky end.” The Type-IIs endonucleases are unique because they generally do not require palindromic recognition sequences and they generally cleave outside of their recognition sites. For example, the Type-IIs endonuclease Earl recognizes and cleaves in the following manner:

| | |
|---|---|
| ↓ | |
| 5′-C-T-C-T-T-C-N-N-N-N-N-3′ | (SEQ ID NO:27) |
| 3′-G-A-G-A-A-G-n-n-n-n-n-5′ | (SEQ ID NO:28) |
| ↑ | |

where the recognition sequence is -C-T-C-T-T-C-, N and n represent complementary, ambiguous base pairs and the arrows indicate the cleavage sites in each strand. As the example illustrates, the recognition sequence is non-palindromic, and the cleavage occurs outside of that recognition site.

Type-IIs endonucleases are generally commercially available and are well known in the art. Specific Type-IIs endonucleases which are useful in the present invention include, e.g., BbvI, BceAI, BfuAI, Earl, AlwI, BbsI, BsaI, BsmAI, BsmBI, BspMI, HgaI, SapI, SfaNI, BsmFI, FokI, and PleI. Other Type-IIs endonucleases that may be useful in the present invention may be found, for example, in the New England Biolabs catalogue. In some embodiments Type-IIs enzymes that generate a recessed 3′ end are particularly useful.

“Adaptor sequences” or “adaptors” are generally oligonucleotides of at least 5, 10, or 15 bases and preferably no more than 50 or 60 bases in length; however, they may be even longer, up to 100 or 200 bases. Adaptor sequences may be synthesized using any methods known to those of skill in the art. For the purposes of this invention they may, as options, comprise primer binding sites, recognition sites for endonucleases, common sequences and promoters. The adaptor may be entirely or substantially double stranded. A double stranded adaptor may comprise two oligonucleotides that are at least partially complementary. The adaptor may be phosphorylated or unphosphorylated on one or both strands. Adaptors may be more efficiently ligated to fragments if they comprise a substantially double stranded region and a short single stranded region which is complementary to the single stranded region created by digestion with a restriction enzyme. For example, when DNA is digested with the restriction enzyme EcoRI the resulting double stranded fragments are flanked at either end by the single stranded overhang 5′-AATT-3′, an adaptor that carries a single stranded overhang 5′-AATT-3′ will hybridize to the fragment through complementarity between the overhanging regions. This “sticky end” hybridization of the adaptor to the fragment may facilitate ligation of the adaptor to the fragment but blunt ended ligation is also possible. Blunt ends can be converted to sticky ends using the exonuclease activity of the Klenow fragment. For example when DNA is digested with PvuII the blunt ends can be converted to a two base pair overhang by incubating the fragments with Klenow in the presence of dTTP and dCTP. Overhangs may also be converted to blunt ends by filling in an overhang or removing an overhang.

An adaptor may be ligated to one or both strands of the fragmented DNA. In some embodiments a double stranded adaptor is used but only one strand is ligated to the fragments. Ligation of one strand of an adaptor may be selectively blocked. Any known method to block ligation of one strand may be employed. For example, one strand of the adaptor can be designed to introduce a gap of one or more nucleotides between the 5′ end of that strand of the adaptor and the 3′ end of the target nucleic acid. Adaptors can be designed specifically to be ligated to the termini produced by restriction enzymes and to introduce gaps or nicks. For example, if the target is an EcoRI digested fragment an adaptor with a 5′ overhang of TTA could be ligated to the AATT overhang left by EcoRI to introduce a single nucleotide gap between the adaptor and the 3′ end of the fragment. Phosphorylation and kinasing can also be used to selectively block ligation of the adaptor to the 3′ end of the target molecule. Absence of a phosphate from the 5′ end of an adaptor will block ligation of that 5′ end to an available 3′OH. For additional adaptor methods for selectively blocking ligation see U.S. Pat. No. 6,197,557 and U.S. Ser. No. 09/910,292 which are incorporated by reference herein in their entirety for all purposes.

Adaptors may also incorporate modified nucleotides that modify the properties of the adaptor sequence. For example, phosphorothioate groups may be incorporated in one of the adaptor strands. A phosphorothioate group is a modified phosphate group with one of the oxygen atoms replaced by a sulfur atom. In a phosphorothioated oligo (often called an “S-Oligo”), some or all of the internucleotide phosphate groups are replaced by phosphorothioate groups. The modified backbone of an S-Oligo is resistant to the action of most exonucleases and endonucleases. Phosphorothioates may be incorporated between all residues of an adaptor strand, or at specified locations within a sequence. A useful option is to sulfurize only the last few residues at each end of the oligo. This results in an oligo that is resistant to exonucleases, but has a natural DNA center.

Methods of ligation will be known to those of skill in the art and are described, for example in Sambrook et at. (2001) and the New England BioLabs catalog both of which are incorporated herein by reference for all purposes. Methods include using T4 DNA Ligase which catalyzes the formation of a phosphodiester bond between juxtaposed 5′ phosphate and 3′ hydroxyl termini in duplex DNA or RNA with blunt and sticky ends; Taq DNA Ligase which catalyzes the formation of a phosphodiester bond between juxtaposed 5′ phosphate and 3′ hydroxyl termini of two adjacent oligonucleotides which are hybridized to a complementary target DNA; E. coli DNA ligase which catalyzes the formation of a phosphodiester bond between juxtaposed 5′-phosphate and 3′-hydroxyl termini in duplex DNA containing cohesive ends; and T4 RNA ligase which catalyzes ligation of a 5′ phosphoryl-terminated nucleic acid donor to a 3′ hydroxyl-terminated nucleic acid acceptor through the formation of a 3′->5′ phosphodiester bond, substrates include single-stranded RNA and DNA as well as dinucleoside pyrophosphates; or any other methods described in the art.

When a fragment has been digested on both ends with the same enzyme or two enzymes that leave the same overhang, the same adaptor may be ligated to both ends. Digestion with two or more enzymes can be used to selectively ligate separate adaptors to either end of a restriction fragment. For example, if a fragment is the result of digestion with EcoRI at one end and BamHI at the other end, the overhangs will be 5′-AATT-3′ and 5′GATC-3′, respectively. An adaptor with an overhang of AATT will be preferentially ligated to one end while an adaptor with an overhang of GATC will be preferentially ligated to the second end.

A genome is all the genetic material of an organism. In some instances, the term genome may refer to the chromosomal DNA. Genome may be multichromosomal such that the DNA is cellularly distributed among a plurality of individual chromosomes. For example, in human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondria DNA. A genomic library is a collection of DNA fragments representing the whole or a portion of a genome. Frequently, a genomic library is a collection of clones made from a set of randomly generated, sometimes overlapping DNA fragments representing the entire genome or a portion of the genome of an organism.

The term “chromosome” refers to the heredity-bearing gene carrier of a living cell which is derived from chromatin and which comprises DNA and protein components (especially histones). The conventional internationally recognized individual human genome chromosome numbering system is employed herein. The size of an individual chromosome can vary from one type to another with a given multi-chromosomal genome and from one genome to another. In the case of the human genome, the entire DNA mass of a given chromosome is usually greater than about 100,000,000 bp. For example, the size of the entire human genome is about 3×10⁹ bp. The largest chromosome, chromosome no. 1, contains about 2.4×10⁸ bp while the smallest chromosome, chromosome no. 22, contains about 5.3×10⁷ bp.

A chromosomal region is a portion of a chromosome. The actual physical size or extent of any individual chromosomal region can vary greatly. The term region is not necessarily definitive of a particular one or more genes because a region need not take into specific account the particular coding segments (exons) of an individual gene.

An allele refers to one specific form of a genetic sequence (such as a gene) within a cell, an individual or within a population, the specific form differing from other forms of the same gene in the sequence of at least one, and frequently more than one, variant sites within the sequence of the gene. The sequences at these variant sites that differ between different alleles are termed “variances”, “polymorphisms”, or “mutations”. At each autosomal specific chromosomal location or “locus” an individual possesses two alleles, one inherited from one parent and one from the other parent, for example one from the mother and one from the father. An individual is “heterozygous” at a locus if it has two different alleles at that locus. An individual is “homozygous” at a locus if it has two identical alleles at that locus.

Capture probes are oligonucleotides that have a 5′ common sequence and a 3′ locus or target specific region or primer. The locus or target specific region is designed to hybridize near a region of nucleic acid that includes a region of interest, for example, near a cytosine of unknown methylation status, so that the locus or target specific region of the capture probe can be used as a primer and be extended through the region of interest to make a copy of the region of interest. The common sequence in the capture probe may be used as a priming site in subsequent rounds of amplification using a common primer or a limited number of common primers. The same common sequence may be present in many or all or the capture probes in a collection of capture probes. Capture probes may also comprise other sequences, for example, tag sequences that are unique for different species of capture probes, and endonuclease recognition sites. In some embodiments the capture probe is designed to hybridize upstream of a position of unknown methylation status and to create a type IIS restriction site that is positioned to cleave between the position of unknown methylation status and the base that is immediately 5′ of the unknown position.

The methylation status of a cytosine is either methylated or unmethylated at position 5. In a diploid organism one copy of a cytosine at a particular location may be methylated while the corresponding copy in the other allele may be unmethylated.

A tag or tag sequence is a selected nucleic acid with a specified nucleic acid sequence. A tag probe has a region that is complementary to a selected tag. A set of tags or a collection of tags is a collection of specified nucleic acids that may be of similar length and similar hybridization properties, for example similar Tₘ. The tags in a collection of tags bind to tag probes with minimal cross hybridization so that a single species of tag in the tag set accounts for the majority of tags which bind to a given tag probe species under hybridization conditions. For additional description of tags and tag probes and methods of selecting tags and tag probes see U.S. Pat. No. 6,458,530 and EP/0799897, each of which is incorporated herein by reference in their entirety.

A collection of capture probes may be designed to interrogate a collection of target sequences. The collection would comprise at least one capture probe for each target sequence to be amplified. There may be multiple different capture probes for a single target sequence in a collection of capture probes, for example, there may be a capture probe that hybridizes to one strand of the target sequence and a capture probe that hybridizes to the opposite strand of the target sequence, these may be referred to as a forward locus or target specific primer and a reverse locus or target specific primer. There also may be two or more capture probes that hybridize at different locations downstream of the target sequence.

A collection of capture probes may be used to amplify a subset of a genome. The collection of capture probes may be initially used to generate a copy of the target sequences in the genomic sample and then the copies may be amplified using common primers. The amplification may be done simultaneously in the same reaction and often in the same tube.

The term “target sequence”, “target nucleic acid” or “target” refers to a nucleic acid of interest. The target sequence may or may not be of biological significance. As non-limiting examples, target sequences may include regions of genomic DNA which are believed to contain one or more cytosines of unknown methylation status, regions of genomic DNA which are believed to contain an imprinted gene, regions of genomic DNA which are believed to contain one a promoter that is regulated by methylation, regions of genomic DNA which are believed to contain a tumor suppressor gene or a promoter region for a tumor suppressor gene, DNA encoding or believed to encode genes or portions of genes of known or unknown function, DNA encoding or believed to encode proteins or portions of proteins of known or unknown function, and DNA encoding or believed to encode regulatory regions such as promoter sequences, splicing signals, polyadenylation signals, etc. The number of sequences to be interrogated can vary, but preferably are from about 1000, 2,000, 5,000, 10,000, 20,000 or 100,000 to 5000, 10,000, 100,000, 1,000,000 or 3,000,000 target sequences.

An “array” comprises a support, preferably solid, with nucleic acid probes attached to the support. Preferred arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 5,800,992, 6,040,193, 5,424,186 and Fodor et al., Science, 251: 767-777 (1991). A plurality of arrays may be simultaneously process in an automated fashion. See, for example U.S. Pat. No. 6,720,149. Each of which is incorporated by reference in its entirety for all purposes.

Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. (See U.S. Pat. Nos. 5,770,358, 5,789,162, 5,708,153, 6,040,193 and 5,800,992, which are hereby incorporated by reference in their entirety for all purposes.) Arrays may be packaged in such a manner as to allow for diagnostic use or can be an all-inclusive device; e.g., U.S. Pat. Nos. 5,856,174 and 5,922,591 incorporated in their entirety by reference for all purposes.

Preferred arrays are commercially available from Affymetrix under the brand name GeneChip® and are directed to a variety of purposes, including genotyping and gene expression monitoring for a variety of eukaryotic and prokaryotic species. (See Affymetrix Inc., Santa Clara and their website at affymetrix.com.) A genotyping array such as the Human Mapping Array 10K Xba 131 may be used to determine the genotype of a collection of SNPs by hybridization. The array contains probes that are specific for each possible allele for a collection of SNPs. Fragments that carry the SNPs are amplified, labeled and hybridized to the array. The presence of a fragment is determined by the hybridization pattern. For additional description of a genotyping array see U.S. provisional patent application No. 60/417,190 filed Oct. 8, 2002.

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See U.S. patent application Ser. No. 08/630,427, filed Apr. 3, 1996.

The term hybridization refers to the process in which two single-stranded nucleic acids bind non-covalently to form a double-stranded nucleic acid; triple-stranded hybridization is also theoretically possible. Complementary sequences in the nucleic acids pair with each other to form a double helix. The resulting double-stranded nucleic acid is a “hybrid.” Hybridization may be between, for example tow complementary or partially complementary sequences. The hybrid may have double-stranded regions and single stranded regions. The hybrid may be, for example, DNA:DNA, RNA:DNA or DNA:RNA. Hybrids may also be formed between modified nucleic acids. One or both of the nucleic acids may be immobilized on a solid support. Hybridization techniques may be used to detect and isolate specific sequences, measure homology, or define other characteristics of one or both strands.

The stability of a hybrid depends on a variety of factors including the length of complementarity, the presence of mismatches within the complementary region, the temperature and the concentration of salt in the reaction. Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) or 100 mM MES, 1 M Na, 20 mM EDTA, 0.01% Tween-20 and a temperature of 25-50° C. are suitable for allele-specific probe hybridizations. In a particularly preferred embodiment hybridizations are performed at 40-50° C. Acetylated BSA and herring sperm DNA may be added to hybridization reactions. Hybridization conditions suitable for microarrays are described in the Gene Expression Technical Manual and the GeneChip Mapping Assay Manual.

Dinucleotide clusters of CpGs or “CpG islands” are present in the promoter and exonic regions of approximately 40% of mammalian genes. By contrast, other regions of the mammalian genome contain few CpG dinucleotides and these are largely methylated. A large number of experiments have shown that methylation of promoter CpG islands plays an important role in gene silencing, genomic imprinting, X-chromosome inactivation, the silencing of intragenomic parasites, and carcinogenesis.

Imprinted genes in the mammalian genome are the genes for which one of the parental alleles is repressed whereas the other one is transcribed. Genetic imprinting, is the result of a mark or imprint carried by a region of the chromosome reflecting the parental origin. Many imprinted genes are located in clusters and are associated with CpG-rich regions that are methylated uniquely on a specific parental chromosome (see, Razin and Cedar (1994) Cell, 77: 473-476; Constancia et al. (1998) 8: 881-900, Reik and Walter (2001) Nature Rev. Genet., 2: 21-32, each of which is incorporated in their entity by reference for all purposes).

CpG islands are regions of the genome containing clusters of CpG dinucleotides. These frequently appear in the 5′ ends of genes. Methylation of CpG islands is known to play a role in transcriptional silencing in higher organisms. The Cs of most CpG dinucleotides in the human genome are methylated, but the Cs in CpG islands are usually unmethylated. Methylation of promoter CpG islands plays an important role in gene silencing, genomic imprinting, X-chromosome inactivation, the silencing of intragenomic parasites, and carcinogenesis.

Imprinted genes in the mammalian genome are the genes for which one of the parental alleles is repressed whereas the other one is transcribed. Genetic imprinting, is the result of a mark or imprint carried by a region of the chromosome reflecting the parental origin. Many imprinted genes are located in clusters and are associated with CpG-rich regions that are methylated uniquely on a specific parental chromosome (see, Razin and Cedar (1994) Cell, 77: 473-476; Constancia et al. (1998) 8: 881-900, Reik and Walter (2001) Nature Rev. Genet., 2: 21-32, each of which is incorporated in their entity by reference for all purposes). Imprinting is another example of epigenetic modification, the expression of the imprinted gene is controlled by patterns of methylation that differ according to the parental origin of the gene. Methods for detecting imprinted genes are disclosed in U.S. Patent Pub No. 20030232353.

An individual is not limited to a human being, but may also include other organisms including but not limited to mammals, plants, bacteria or cells derived from any of the above.

(C.) Array Based Methylation Analysis

Several methods have been described for identification of altered methylation sites in genomic samples including cancer cells. Methods include, for example, restriction landmark genomic scanning (Hatada et al. Proc. Natl. Acad. Sci. USA 88: 9523-9527, 1992 and Kawai et al., Mol. Cell. Biol. 14: 7421-7427, 1994), and methylation-sensitive arbitrarily primed PCR (Gonzalgo et al., Cancer Res. 57: 594-599, 1997 and Liang et al. Methods 27: 150-155, 2002). Changes in methylation patterns at specific CpG sites have been monitored by digestion of genomic DNA with methylation-sensitive restriction enzymes followed by Southern analysis of the regions of interest (digestion-Southern method). Another method for analyzing changes in methylation patterns involves a PCR-based process that involves digestion of genomic DNA with methylation-sensitive restriction enzymes prior to PCR amplification (Singer-Sam et al., Nucl. Acids Res. 18: 687, 1990). Methylation-sensitive amplification polymorphism is another technique based on methylation specific polymorphisms (Peraza-Exheverria et al., Plant Sci. 161: 359-367, 2001). Other methods based on methylation sensitive enzymes include, for example, methylated CpG island amplification (MCA) (Toyota et al. Cancer Res. 59: 2307-2312, 1999) and the methods of Brock et al. Gene 240: 269-277, 1999).

Several methods for analysis of DNA methylation patterns and 5-methylcytosine distribution involve bisulfite treatment of the DNA (Frommer et al., Proc. Natl. Acad. Sci. USA 89: 1827-1831, 1992). Bisulfite treatment of DNA distinguishes methylated from unmethylated cytosines, and can be detected by sequencing after treatment. Other bisulfite based methods for methylation analysis include methylation-specific PCR (MSP) (Herman et al. Proc. Natl. Acad. Sci. USA 93: 9821-9826, 1992); restriction enzyme digestion of PCR products amplified from bisulfite-converted DNA (Sadri and Hornsby, Nucl. Acids Res. 24: 5058-5059, 1996; and Xiong and Laird, Nucl. Acids. Res. 25: 2532-2534, 1997); methylation sensitive single nucleotide primer extension (Ms-SNuPE) (Gonzalgo and Jones, Nucl. Acids Res. 25, 2529-2531 (1997); and SNuPE with ion pair reverse phase HPLC (El-Maarri et al. Nucl. Acids Res. 30: 225 (2002).

Methods are disclosed for high throughput analysis of the methylation status of a plurality of different cytosines simultaneously. In some embodiments methlylation specific amplification methods are combined with arrays of oligonucleotide probes. Methods are disclosed for determining the methylation status of one or more cytosines in the starting sample by hybridizing the amplified sample to an array of probes. Methods are disclosed for rapid assessment of the methylation status of a plurality of cytosines simultaneously.

Methods are disclosed for using arrays of oligonucleotides to determine the presence or absence of methylation in a nucleic acid sample. In preferred embodiments the arrays comprise a plurality of oligonucleotides of known sequence that are present at known locations or features on a solid support. Hybridization of a nucleic acid that is complementary to the oligonucleotide probes of a feature can be detected to indicate the presence of a particular sequence in a sample. Arrays that may be useful for the methods include, for example, genotyping arrays, resequencing arrays, expression arrays, tiling arrays, whole genome arrays and custom arrays that are designed to detect methylation at specific locations in a genome. Specific examples of arrays that may be used include arrays available from Affymetrix, Inc., including the 10K and 100K Mapping Arrays, CustomSeq arrays, expression arrays such as the Human Genome U133 Plus 2.0 array and tiling arrays such as the arrays described in Kampa et al. Genome Res. 2004 March; 14 (3): 331-42, Cawley et al. Cell 2004 Feb. 20; 116 (4): 499-509 and Kapranov et al. Science 2002 May 3; 296 (5569): 916-9, each of which is incorporated by reference in its entirety. In some embodiments methods that use an array of tag probes, for example, the Affymetrix GenFlex and Tag3 array, may be used. In some embodiments an array of beads may be used where each bead comprises a tag or tag probe sequence.

In a preferred embodiment genomic DNA is treated so that methylated and unmethylated DNA regions are differentially amplified. In some embodiments a nucleic acid sample is enriched for fragments that contain only unmethylated cytosines relative to fragments that contain one or more methyl cytosines. For example, in some embodiments fragments of DNA are amplified so that the fragments that contain only unmethylated cytosines are enriched in the amplified product relative to fragments that contain one or more methyl cytosines. In another example, fragments that contain methyl cytosine may be preferentially degraded chemically or enzymatically. In another embodiment fragments that contain methyl cytosine are enriched in the sample relative to unmethylated fragments. In many embodiments the enriched fragments are labeled and hybridized to an array and hybridization is detected. In some embodiments the presence of hybridization is an indication that a fragment is present and absence of hybridization is an indication that a fragment is absent. In some embodiments the amount of hybridization is an indication of the amount of methylation.

The methods are particularly well suited for high throughput analysis of the methylation status of cytosines. High throughput methods of array analysis are described in U.S. Patent Publication No. 20030124539 and in U.S. Pat. No. 6,720,149. In a single experiment more than 100, 1000, 10,000, or 100,000 different cytosine positions in a sample may be analyzed for methylation status. Many samples may be processed in parallel. Samples from more than 10, 100, or 1000 individuals may be processed in parallel. The methods may employ for example, microtitre plates, automated methods of sample preparation and sample handling and computer methods to track samples and analyze data.

In some embodiments, modification of methylated cytosine may be done by treatment with sodium bisulfite. See Frommer et al. Proc. Natl. Acad. Sci. USA 89: 1827-1831, 1992 and Clark et al. Nucleic Acids Res 1994 Aug. 11; 22 (15): 2990-7. Sodium bisulfite modification converts unmethylated cytosine to uracil through a three-step process. If the cytosine is methylated it will remain a cytosine. Methylated cytosines remain cytosines and a C:G basepair is maintained in subsequent amplification steps while an unmethylated C becomes a U and results in a T:A base pair following amplification. The methylated and unmethylated cytosines can be distinguished by any method that is capable of differentially detecting a uracil and a cytosine.

The sequence at the position being interrogated for methylation can be determined and if it is still a C then the position was methylated. If a T is present then the position was unmethylated. Methods that may be used to detect the base present at a SNP position may be used. For example, genotyping methods based on single base extension (SBE) or an oligo ligation assay (OLA) may be used to detect the presence of an A or G on one strand or a T or C on the other strand. Hybridization to sequence specific oligonucleotides may also be used, for example, sets of probes designed to hybridize specifically to either the A/T or G/C base pair. The probes may be designed to hybridize to one strand or to both strands. The probes may be similar to probe sets designed to hybridize specifically to one or the other allele of a biallelic SNP.

In some embodiments a position may be partially methylated in the genome. Partial modification would be expected to result in a mixture of T and C at the position being interrogated. Hybridization would be observed to both the T specific probes and the C specific probes, similar to detection of a heterozygous SNP. Relative amounts of hybridization may be used to determine the relative amount of methylation.

In another embodiment methylation status is determined after sodium bisulfite treatment through extension of a locus specific primer. The locus specific primer may then be detected by hybridization to an array. In a preferred embodiment the locus specific primer has a common sequence that may be used for priming amplification and a locus-specific region. The primer may be extended, for example, using ddNTP mini-sequencing or single base extension. A locus specific primer may be designed for each CpG site to be analyzed. A plurality of locus specific primers, each designed to assay a different CpG site may be designed and used simultaneously in the same reaction. Each of the primers may have a different locus specific region and the same common sequence so that a single primer may be used for amplification. SBE may be followed by hybridization to an array of tag probes. The hybridization pattern is determined and analyzed to determine the methylation status of selected cytosines.

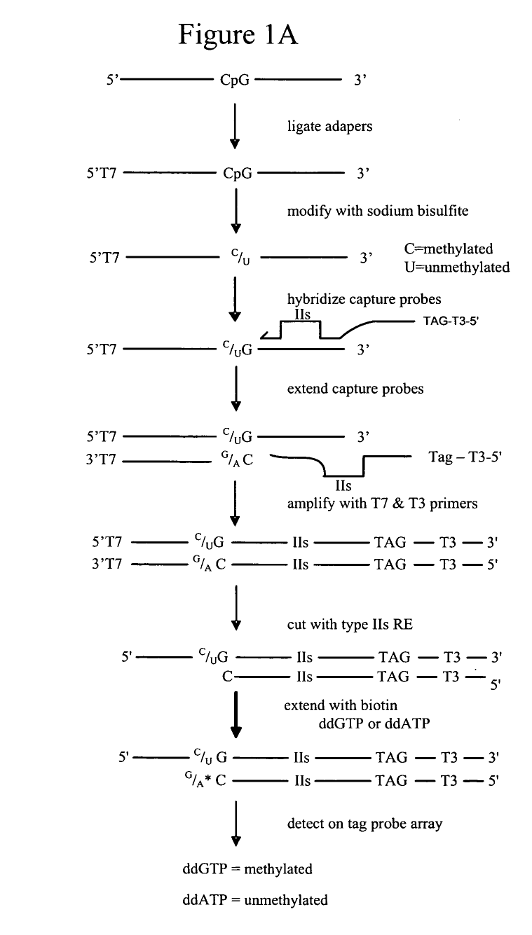

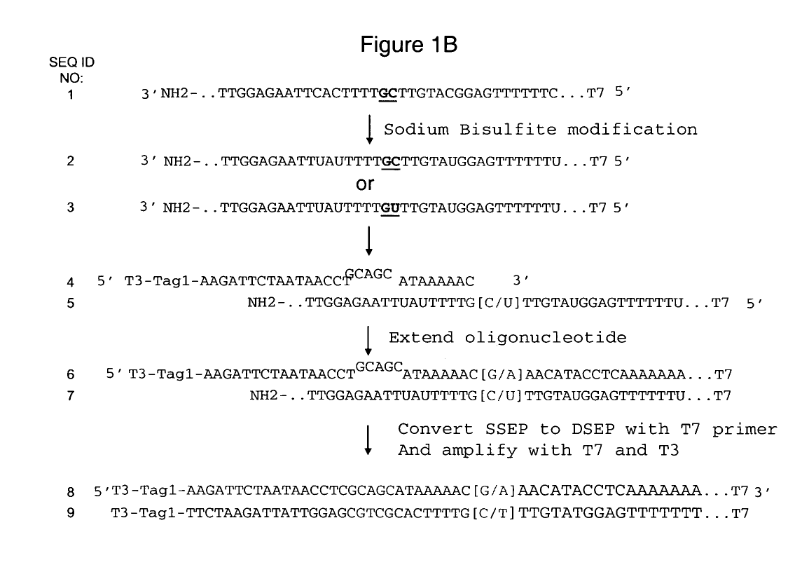

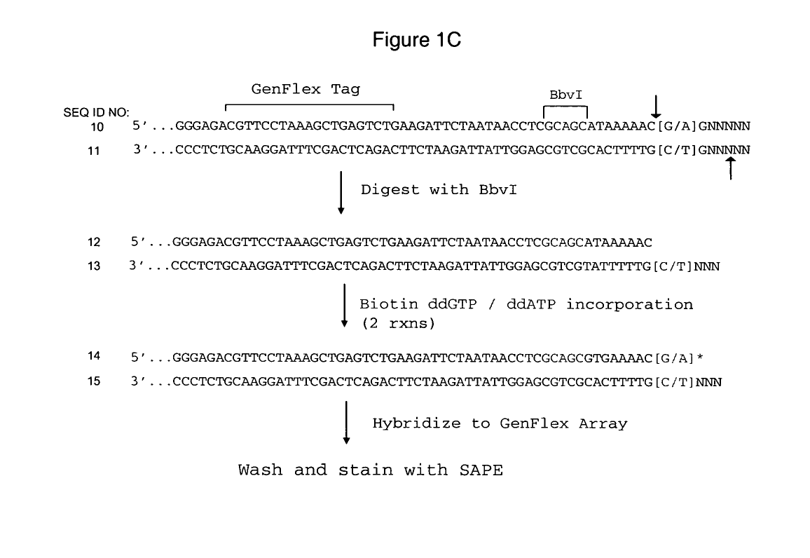

In many embodiments, a nucleic acid sample is fragmented, ligated to an adaptor with a 5′ first common sequence (FIG. 1A). The fragments are modified with sodium bisulfite. Locus specific primers that hybridize near the selected cytosine and have a 5′ second common sequence, a tag sequence and a recognition site for a Type IIS restriction enzyme (FIG. 1B) are hybridized to the fragments and extended, generating a double stranded extension product. The double stranded extension product is flanked by the first and second common sequences and can be amplified using primers to these sequences. The first and second common sequences may be a promoter sequence for a phage promoter, such as T7 or T3. The amplified fragments are digested with the Type IIS restriction enzyme (see FIGS. 1A and 1C). The enzyme recognition site is positioned so that cleavage occurs immediately 5′ of the position being interrogated. The strand can then be extended by a single base corresponding to the base being interrogated. In one embodiment the strand extended is the strand opposite the strand containing the C being interrogated and a G is incorporated if the C was methylated and remained unmodified or an A is incorporated if the C was unmethylated and modified. Incorporation of primarily G's indicates that both chromosomal copies were methylated; incorporation of primarily A's indicates that both chromosomal copies were unmethylated; and incorporation of approximately equal levels of A and G indicates that one chromosomal copy may have been methylated while the other remained unmethylated, suggesting that the locus may be an imprinted locus. In another embodiment the opposite strand is interrogated and either a C or a T is incorporated.

When the locus specific primer is extended a G will be incorporated in the interrogation position (opposite the C being interrogated) if the C was methylated and an A will be incorporated in the interrogation position if the C was unmethylated. When the double stranded extension product is amplified those C's that were converted to U's and resulted in incorporation of A in the extended primer will be replaced by T's during amplification. Those C's that were not modified and resulted in the incorporation of G will remain as C. The base pair at the interrogation position will either be an A/T, indicating an unmethylated C or a G/C indicating a methylated C.

In one embodiment ddATP and ddGTP are used for extension so only a single A or G will be added. The ddATP and ddGTP may be labeled with differentially detectable labels and used in the same reaction or they may be labeled with the same detectable label, biotin for example, and separated into individual reactions.

In one embodiment the labeled extended products are detected by hybridization to an array of tag probes. The probes of the array may be complementary to tags in the locus specific primers. For additional description of tags and tag probes, see, U.S. Pat. No. 6,458,530 and Ser. No. 09/827,383 which are herein incorporated by reference. In one embodiment the tags used are complementary to the tag probes on the GenFlex array, available from Affymetrix, Inc. If the extension products are differentially labeled the extension reaction may be hybridized to the same array. Alternatively, if the extension products are labeled with the same label they may be hybridized to separate arrays.

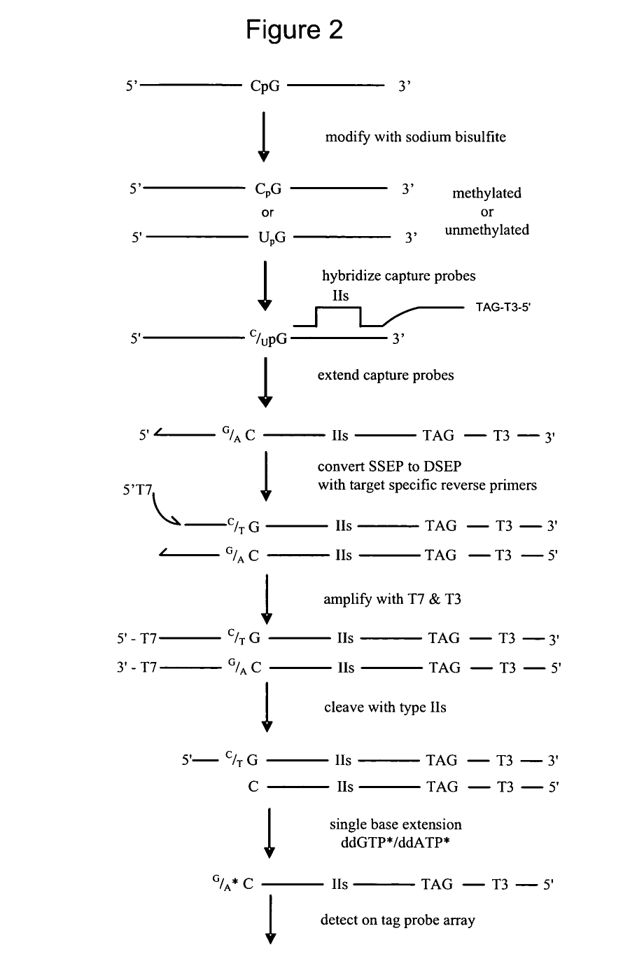

In another embodiment (FIG. 2) adaptors are not ligated to the fragmented nucleic acid and conversion of the single stranded extension product to a double stranded extension product is done by using locus specific reverse primers. Genomic DNA is fragmented and subjected to methylation specific modification, for example, with sodium bisulfite. Capture probes are hybridized to the modified fragments and extended through the cytosine position of interest to generate single stranded extension probes. Target specific reverse primers are hybridized to the single stranded extension probes and extended to generate double stranded extension probes. The target specific reverse primers comprise a common priming sequence located 5′ of the locus specific sequence. The double stranded extension products may be amplified using common sequence primers. The amplified products are then digested with a type IIS restriction enzyme which cleaves between the interrogation position and the base that is just 5′ of the interrogation position. The fragment is then extended by one base corresponding to the interrogation position. The base that is incorporated is determined by hybridization to an array of tag probes that are complementary to the tag sequences in the capture probes. In another embodiment the type IIs recognition site is introduced in the target specific reverse primers. A plurality of cytosines may be interrogated using a plurality of capture probes and a plurality of target specific reverse primers. Each probe in the plurality of capture probes and each primer in the plurality of target specific reverse primers may be specific for a target sequence.

Capture probes may be attached to a solid support so that they have a free 3′ end. In some embodiments the capture probes are synthesized on a solid support. A plurality of a single species of capture probes may be synthesized at a discreet location on an array and may form a discrete feature of an array. Each feature of the array may contain a different species of locus specific capture probe. The capture probes may be extended while attached to the array or after release from the array. Any suitable solid support known in the art may be used, for example, arrays, beads, microparticles, microtitre dishes and gels may be used. In some embodiments the capture probes are synthesized on an array in a 5′ to 3′ direction.

Information about the region of interest can be determined by analysis of the hybridization pattern. The amplified sample may be analyzed by any method known in the art, for example, MALDI-TOF mass spec, capillary electrophoresis, OLA, dynamic allele specific hybridization (DASH) or TaqMan® (Applied Biosystems, Foster City, Calif.). For other methods of analyses see Syvanen, Nature Rev. Gen. 2: 930-942 (2001) which is herein incorporated by reference in its entirety.

In another embodiment regions that contain possible methylation sites are interrogated for methylation using resequencing. The genomic sample is modified with sodium bisulfite. The regions of interest are amplified using locus specific PCR primers and long range PCR. The amplicons are fragmented and labeled and hybridized to a resequencing array. The hybridization pattern is analyzed to determine if the CpG's are methylated.

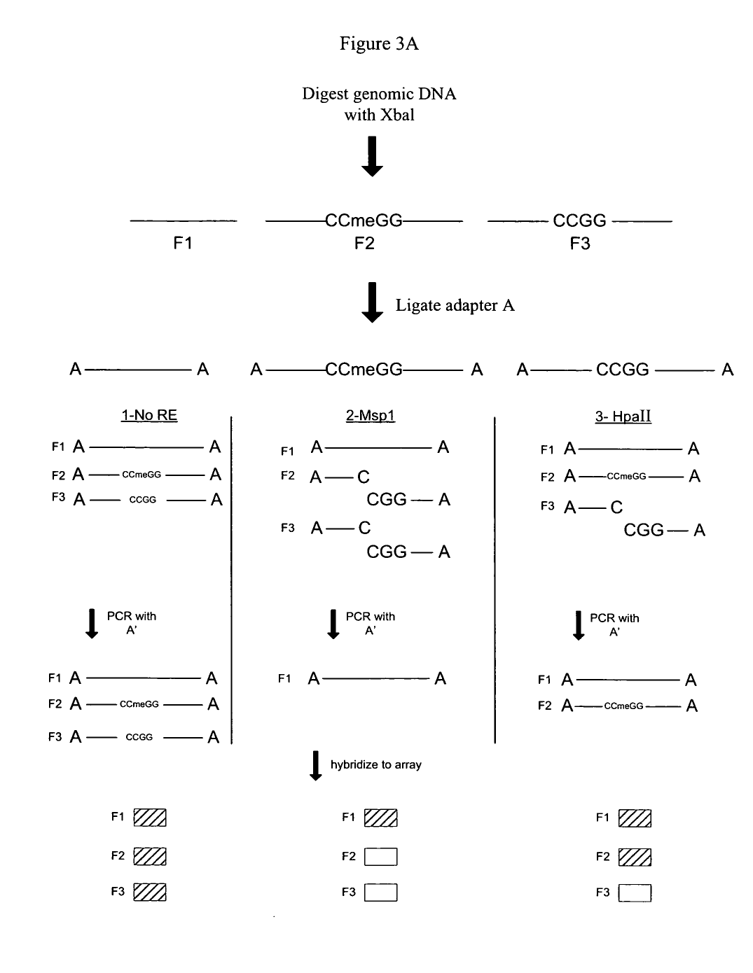

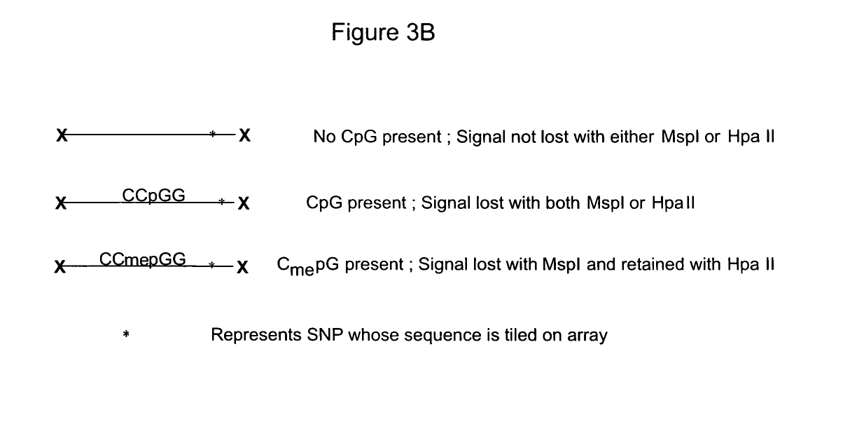

In another embodiment the methylation status of a cytosine is analyzed using differential digestion. In one preferred embodiment genomic DNA is subjected to restriction digestion with two restriction enzymes that recognize the same recognition site but are differentially sensitive to methylation, see, FIG. 3. In one embodiment HpaII and MspI are used and the cytosine is part of a CpG dinucleotide. HpaII and MspI are isoschizomers which cleave at recognition site CCGG (see, New England Biolab Catalogue, which is incorporated herein by reference in its entirety). Cleavage by HpaII is blocked by methylation while MspI cleaves independent of methylation. A genomic DNA sample is digested with a restriction enzyme and adaptors are ligated to the fragments to generate a population of adaptor-modified fragments. The sample is divided into three fractions. One fraction is fragmented with Hpa II, a second fraction is fragmented with MspI and the final fraction is left untreated. Each of the fractions is then amplified using primers to the adaptors. The amplified products are then hybridized to a array of probes designed to interrogate the presence or absence of specific fragments, for example, the array disclosed in U.S. patent application Ser. Nos. 10/264,945, 09/916,135 and 60/417,190 each of which is incorporated herein by reference. Fragments that have the CCGG recognition site will either be cleaved in both the MspI and HpaII fractions if the CpG is unmethylated or will be cleaved in the MspI fraction but not the HpaII fraction if the CpG is methylated. After cleavage the samples are amplified using primers to the adaptor sequences. If a fragment has been cleaved by MspI or HpaII the fragment will not be amplified in the PCR reaction because the resulting fragments will have the adaptor sequence, and therefore the priming site, only on one end.

Possible outcomes for a given fragment that is interrogated by the array are as follows: if the fragment does not have the CCGG recognition site it should be present in each of the three fractions (F1 in FIG. 3A); if the fragment has the CCGG site and the CpG is methylated in at least some of the fragments it should be present in the undigested sample, absent from the MspI sample and present in the HpaII digested samples (F2 in FIG. 3A); if the fragment has the CCGG and the CpG is unmethylated it should be present in the undigested sample, but absent in the MspI digested sample and absent in the HpaII digested sample (F3 in FIG. 3A). See also U.S. Pat. No. 6,605,432 which discloses methods of detecting DNA methylation. Additional methods of analysis of methylation are disclosed in U.S. Provisional Application No. 60/544,844 filed Feb. 13, 2003 and 60/526,336 filed Dec. 2, 2003.

In a preferred embodiment in silico digestion methods can be used to predict which fragments will be present in the amplified sample. For example, if the first digestion is with XbaI then fragments that are in the size range to be amplified, approximately 200 to 2000 bp in a preferred embodiment, and that contain the CCGG recognition site will be interrogated. An array may be designed to detect these fragments or a subset of these fragments. In one embodiment the probes of the array may be further designed to interrogate a subset of these fragments, for example, those fragments that contain promoter regions.

Generally, the invention provides methods for highly multiplexed locus specific amplification of nucleic acids that preserves information about the methylation status of cytosines in the starting sample and determination of methylation status. In some embodiments the invention combines the use of capture probes that comprise a common sequence and a locus-specific region with adaptor-modified sample nucleic acid; the adaptor comprises a second common sequence. The capture probes are extended to produce copies of the sample DNA that contain common priming sequences flanking the target sequence. The copies are amplified with a generic set of primers that recognize the common sequences. The amplified product may be analyzed by hybridization to an array of probes.

In one embodiment the steps of the invention comprise: generating capture probes; digesting a nucleic acid sample; ligating adaptors to the fragmented sample; mixing the fragments and the capture probes under conditions that will allow hybridization of the fragments and the capture probes; extending the capture probes in the presence of dNTPs and polymerase; amplifying the extended capture probes; and detecting the presence or absence of target sequences of interest.

In some embodiments a collection of target sequences is analyzed. A plurality of capture probes is designed for a plurality of target sequences. In some embodiments target sequences contain or are predicted to contain a methylated cytosine which may be part of a CpG dinucleotide. The cytosine may be, for example, in the promoter region of a gene whose expression may be regulated by methylation. A collection of capture probes may be designed so that each capture probe hybridizes near a cytosine of interest. The capture probes hybridize to one strand of the target sequence and can be extended through the region where the cytosine of interest is located so that the extension product comprises a copy of one strand of the region surrounding and including the cytosine.

Many amplification methods are most efficient at amplification of smaller fragments. For example, PCR most efficiently amplifies fragments that are smaller than 2 kb (see, Saiki et al. 1988). In one embodiment capture probes and fragmentation conditions are selected for efficient amplification of a selected collection of target sequences. The size of the amplified fragments is dependent on where the target specific region of the capture probe hybridizes to the target sequence and the 5′ end of the fragment strand that the capture probe is hybridized to. In some embodiments of the present methods capture probes and fragmentation methods are designed so that the target sequence of interest can be amplified as a fragment that is, for example, less than 20,000, 2,000, 800, 500, 400, 200 or 100 base pairs long. The capture probe can be designed so that the 3′ end of the target specific region hybridizes to the base that is just 3′ of a position to be interrogated in the target sequence. More than one capture probe may be designed for a target sequence to analyze different cytosines that are present in a single target fragment. When the sample is fragmented with site specific restriction enzymes the length of the fragments will also depend on the position of the nearest recognition site for the enzyme or enzymes used for fragmentation. A collection of target sequences may be selected based on proximity to restriction sites.

In some embodiments target sequences are selected for amplification and analysis based on the presence of a cytosine of interest, such as a cytosine in a CpG dinucleotide or CpG island, and proximity to a cleavage site for a selected restriction enzyme. For example, fragments comprising a cytosine of interest that is within 200, 500, 800, 1,000, 1,500, 2,000 or 20,000 base pairs of a restriction site, such as, for example, an EcoRI site, a BglI site, an XbaI site or any other restriction enzyme site may be selected to be target sequences in a collection of target sequences and capture probes may be designed to interrogate one or more cytosines in the target sequence. In another method a fragmentation method that randomly cleaves the sample into fragments that are 30,100, 200, 500 or 1,000 to 100, 200, 500, 1,000 or 2,500 base pairs on average may be used. A unique capture probe is designed for each cytosine to be interrogated.

In many embodiments of the present methods one or more enrichment step may be included to generate a sample that is enriched for extended capture probes prior to amplification with common sequence primers. In some embodiments it is desirable to separate extended capture probes from fragments from the starting nucleic acid sample, adaptor-ligated fragments, adaptor sequences or non-extended capture probes, for example. In one embodiment the capture probes are extended in the presence of a labeled dNTP, for example dNTPs labeled with biotin. The labeled nucleotides are incorporated into the extended capture probes and the labeled extended capture probes are then separated from non-extended material by affinity chromatography. When the label is biotin the labeled extended capture probes can be isolated based on the affinity of biotin for avidin, streptavidin or a monoclonal anti-biotin antibody. In one embodiment the antibody may be coupled to protein-A agarose, protein-A sepharose or any other suitable solid support known in the art. Those of skill in the art will appreciate that biotin is one label that may be used but any other suitable label or a combination of labels may also be used, such as fluorescein which may be incorporated in the extended capture probe and an anti-fluorescein antibody may be used for affinity purification of extended capture probes. Other labels such as, digoxigenin, Cyanine-3, Cyanine-5, Rhodamine, and Texas Red may also be used. Antibodies to these labeling compounds may be used for affinity purification. Also, other haptens conjugated to dNTPs may be used, such as, for example, dinitrophenol (DNP).

In another embodiment extension products may be enriched by circularization followed by digestion with a nuclease such as Exonuclease VII or Exonuclease III. The extended capture probes may be circularized, for example, by hybridizing the ends of the extended capture probe to an oligonucleotide splint so that the ends are juxtaposed and ligating the ends together. The splint will hybridize to the common sequences in the extended capture probe and bring the 5′ end of the capture probe next to the 3′ end of the capture probe so that the ends may be ligated by a ligase, for example DNA Ligase or Ampligase Thermostable DNA. See, for example, U.S. Pat. No. 5,871,921 which is incorporated herein by reference in its entirety. The circularized product will be resistant to nucleases that require either a free 5′ or 3′ end.

A variety of nucleases may be used in one or more of the embodiments. Nucleases that are commercially available and may be useful in the present methods include: Mung Bean Nuclease, E. Coli Exonuclease I, Exonuclease III, Exonuclease VII, T7 Exonuclease, BAL-31 Exonuclease, Lambda Exonuclease, RecJf, and Exonuclease T. Different nucleases have specificities for different types of nucleic acids making them useful for different applications. Exonuclease I catalyzes the removal of nucleotides from single-stranded DNA in the 3′ to 5′ direction. Exonuclease I degrades excess single-stranded primer oligonucleotide from a reaction mixture containing double-stranded extension products. Exonuclease III catalyzes the stepwise removal of mononucleotides from 3′-hydroxyl termini of duplex DNA. A limited number of nucleotides are removed during each binding event, resulting in coordinated progressive deletions within the population of DNA molecules. The preferred substrates are blunt or recessed 3′-termini, although the enzyme also acts at nicks in duplex DNA to produce single-strand gaps. The enzyme is not active on single-stranded DNA, and thus 3′-protruding termini are resistant to cleavage. The degree of resistance depends on the length of the extension, with extensions 4 bases or longer being essentially resistant to cleavage. This property can be exploited to produce unidirectional deletions from a linear molecule with one resistant (3′-overhang) and one susceptible (blunt or 5′-overhang) terminus. Exonuclease VII is a single-strand directed enzyme with 5′ to 3′- and 3′ to 5′-exonuclease activities making it the only bi-directional E. coli exonuclease with single-strand specificity. The enzyme has no apparent requirement for divalent cation, and is fully active in the presence of EDTA. Initial reaction products are acid-insoluble oligonucleotides which are further hydrolyzed into acid-soluble form. The products of limit digests are small oligomers (dimers to dodecamers). For additional information about nucleases see catalogues from manufacturers such as New England Biolabs, Beverly, Mass.

In some embodiments one of the primers added for PCR amplification is modified so that it is resistant to nuclease digestion, for example, by the inclusion of phosphorothioate. Prior to hybridization to an array one strand of the double stranded fragments may be digested by a 5′ to 3′ exonuclease such as T7 Gene 6 Exonuclease.

In some embodiments the nucleic acid sample, which may be, for example, genomic DNA, is fragmented, using for example, a restriction enzyme, DNase I or a non-specific fragmentation method such as that disclosed in U.S. Pat. No. 6,495,320, which is incorporated herein by reference in its entirety.

In some embodiments the amplified products are analyzed by hybridization to an array of probes attached to a solid support. In some embodiments the array of probes is designed to interrogate the presence or absence of a collection of target sequences. The array of probes may interrogate, for example, from 1,000, 5,000, 10,000 or 100,000 to 2,000, 5,000, 10,000, 100,000, 1,000,000 or 3,000,000 different target sequences. Any array of probes that can be used to detect the presence or absence of a target sequence may be used. The array may be, for example, designed to interrogate target sequences containing SNPs and the array of probes may be designed to interrogate the allele or alleles present at one or more polymorphic location. See, for example, U.S. patent application Ser. Nos. 09/916,135, 10/264,945, 10/681,773 and 60/417,190 which are each incorporated herein by reference in their entirety.

In a preferred embodiment the array is designed to interrogate target sequences containing sites of potential methylation following treatment with sodium bisulfite which converts unmethylated cytosines to uracil. Probes are designed to be perfectly complementary to specific regions that contain sites of potential methylation and in a preferred embodiment probe design takes into account modification of surrounding bases. For example, to interrogate a particular CpG for methylation using bisulfite modification a probe may be designed to be perfectly complementary to the methylated.

In another embodiment an array of probes that are complementary to tag sequences present in the capture probes is used to interrogate the target sequences. In some embodiments the amplified targets are analyzed on an array of tag sequences, for example, the Affymetrix GenFlex® array (Affymetrix, Inc., Santa Clara, Calif.). In this embodiment the capture probes comprise a tag sequence that is unique for each species of capture probe and tag probes of the array are complementary to the tag sequence. A detectable label that is indicative of the methylation status of the cytosine present at the site of interest is associated with the tag. The labeled tags are hybridized to the one or more arrays and the hybridization pattern is analyzed. The base that is incorporated in the capture probe is indicative of the methylation status, for example, in FIG. 1 if a G is incorporated the methylation status of the cytosine is methylated and if an A is incorporated the methylation status of the cytosine is unmethylated. If there is a mixture of A and G incorporated one copy of the target sequence may be methylated while the other is unmethylated, possibly indicating an imprinted gene.

The methylation status of, for example, from 100, 500, 1,000, 5,000, 10,000 or 100,000 to 200, 2,000, 5,000, 10,000, 100,000, 1,000,000 or 3,000,000 different cytosines may be analyzed simultaneously. Anaylsis of multiple cytosines may be done in a single reaction and using a single tube.

In another embodiment kits that are useful for the present methods are disclosed. In one embodiment a kit for amplifying a collection of target sequences is disclosed. The kit may comprise one or more of the following: a collection of capture probes as disclosed, one or more adaptor, one or more generic primers for common sequences, one or more restriction enzymes, buffer, one or more polymerase, a ligase, buffer, dNTPs, ddNTPs, and one or more nucleases. The restriction enzyme of the kit may be a type-IIs enzyme. The capture probes may be attached to a solid support. The kit may comprise an array designed to interrogate the methylation of a plurality of different pre-selected cytosines.

In one embodiment methylation is detected at pre-selected cytosines using methylation specific modification and complexity reduction using adaptor mediated ligation followed by detection on a microarray comprising methylation specific oligonucleotides that are perfectly complementary to a region surrounding and including a methylation site to be interrogated. There are at least two probes for each methylation site, a first that is complementary to the product resulting from sodium bisulfite if the cytosine is not methylated and the second complementary to the product resulting from sodium bisulfite modification if the cytosine is methylated.

In one embodiment genomic DNA is subjected to sodium bisulfite treatment, fragmented with one or more restriction enzymes, ligated to one or more adaptors and amplified using the whole genome sampling assay described in U.S. Pat. No. 6,361,947 and U.S. patent application Ser. Nos. 09/916,135, 10/740,230 and 10/442,021, and U.S. Patent Publication Nos. U.S. 20030036069 and 20040072217 A1.

Amplification products may be fragmented, for example, by DNase treatment, and labeled, for example, using terminal transferase (TdT). The labeled fragments are hybridized to an array of probes. The probes are designed to detect the presence or absence of methylation at specific cytosines like a SNP. For each cytosine to be interrogated for methylation the array has a first probe set that is specific for the presence of the C base at the interrogation position and a probe set that is specific for the presence of a T base at the interrogation position. The probe sets are analogous to the probe sets of the 10K Mapping Array except instead of interrogating the genotype of a SNP the probe sets interrogate the presence of a C or a T at a cytosine of interest.

The steps of fragmenting, ligating adaptors and modifying with the methylation specific modifier may be done in a different order in some embodiments. In one embodiment the nucleic acid sample is first fragmented, then adaptors are ligated to the fragments and the adaptor ligated fragments are modified. In this embodiment the adaptors would be subject to modification so unmethylated C's would be converted to U's. During the amplification step with a common primer the primer may be designed to take this into consideration. For example, if the adaptor sequence is 3′-AACGTG-5′ and the C is not methylated it will be modified and the sequence will become 3′-AAUGTG-5′. The primer for amplification may be 5′-TTACAC-3′. In another embodiment the adaptors are modified to contain 5-methyl cytosine so that the sequence will not be modified. In another embodiment the nucleic acid sample is modified before the adaptors are ligated. The nucleic acid may be modified before or after fragmentation.

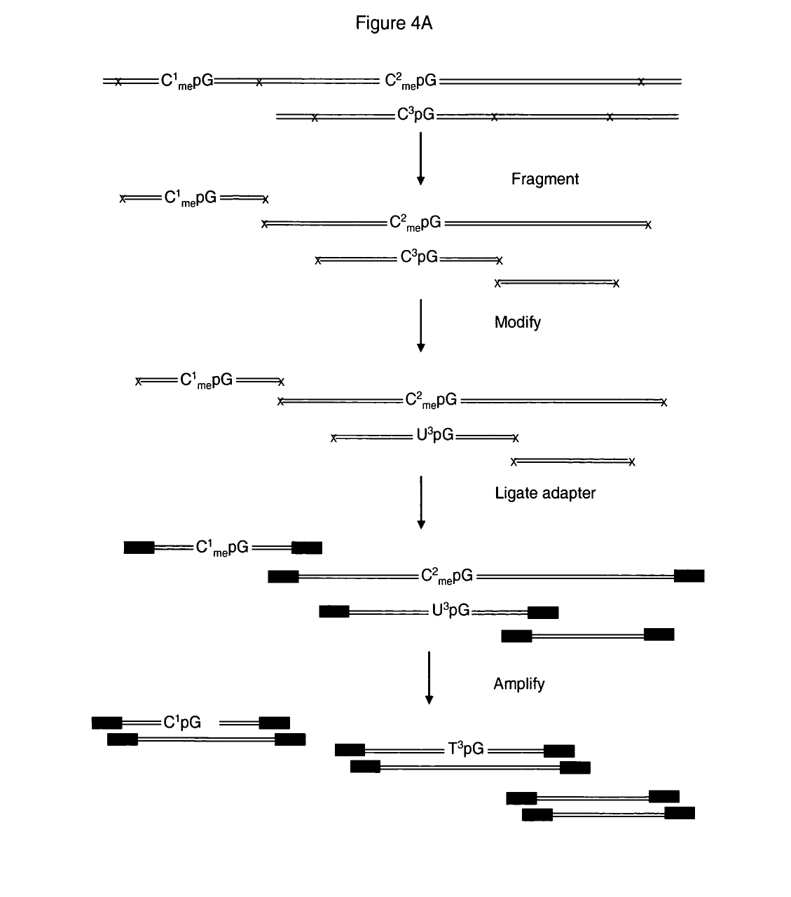

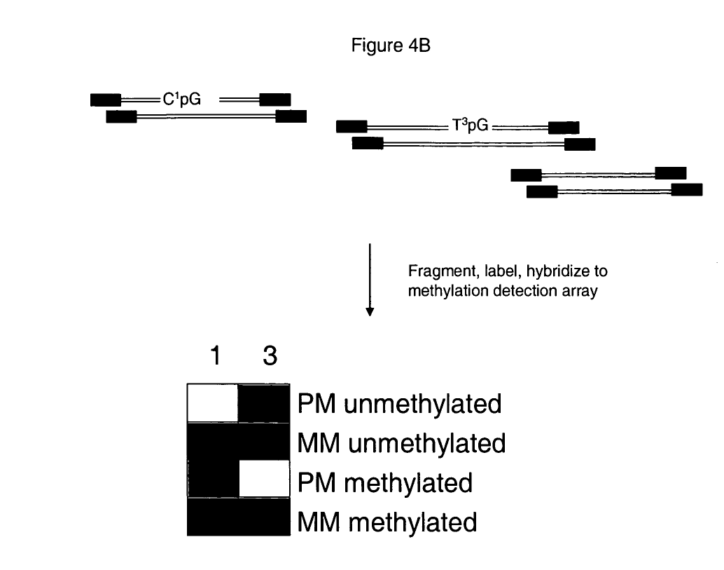

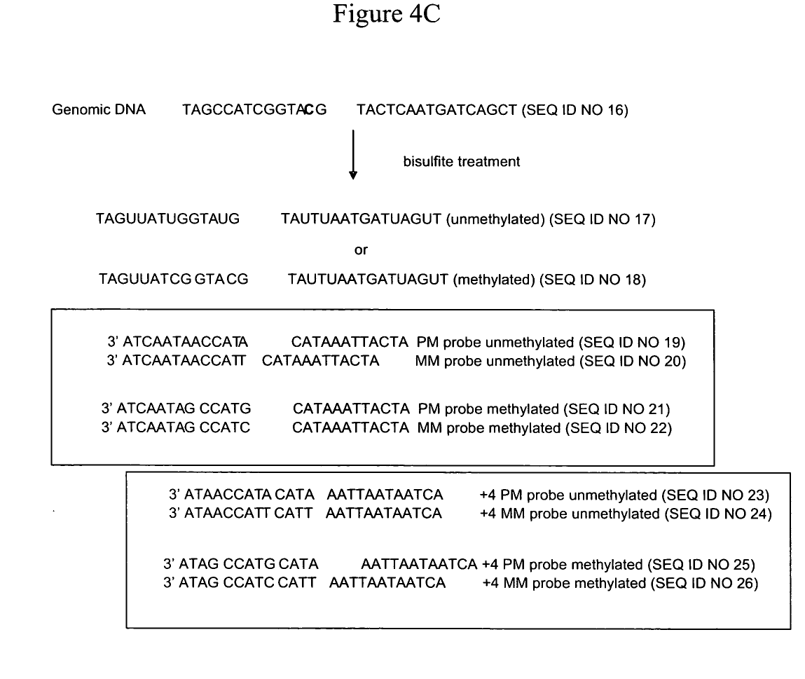

FIG. 4A shows a method of sample preparation, showing three different CpG sites indicated as 1, 2 and 3. Genomic DNA is fragmented with, for example a restriction enzyme and modified with sodium bisulfite. Adaptors are ligated to the ends of the fragments and fragments are amplified using a single primer that is complementary to the adaptor sequence. Fragments of a limited size range are most efficiently amplified and are enriched in the product relative. Fragments that are less than about 200 base pairs are not efficiently amplified because of complementarity between the ends of a fragment, resulting in pan handle formation. Fragments that are larger than about 2,000 or 2,500 base pairs are not efficiently amplified under standard PCR conditions. In the example shown in FIG. 4 the fragments containing sites 1 and 3 are amplified. The fragment containing site 2 is not amplified because it is longer than about 2,500 base pairs. The cytosine in site 1 is methylated so it is not modified by bisulfite treatment while the cytosine in site 3 is modified to a uracil which is changed to a T:A base pair during amplification. FIG. 4B shows detection on an array designed to detect selected sites of possible methylation. There is a probe set for site 1 and a probe set for site 2 but no probe set for site 3 because in silico digestion predicted that sites 1 and 3 would be amplified efficiently but not site 3. The remaining fragment that does not contain a possible methylation site is also not interrogated by the array. The array contains probes to interrogate methylation of CpG sites that are predicted to be present after digestion with a specific enzyme or enzymes and amplification. Absence of hybridization is shown as a filled box, so hybridization is observed for the cite I in the PM unmethylated probe and for cite 3 for the PM methylated probe. In silico digestion methods can be used to identify CpG's that fit a specified set of criteria and probes may be designed to interrogate CpG's in that set. FIG. 4C shows an example of how probes may be designed. Probe and primer design may take into account the results of sodium bisulfite modification.

### EXAMPLES

### Example 1

### Analysis of 5-methyl C Using Multiplex Runoff Amplification

Genomic DNA may be digested with XbaI and ligated to an adaptor containing T7 promoter sequence as a priming site. The adaptor-ligated genomic DNA may be modified with sodium bisulfite followed by purification over a Qiagen (Valencia, Calif.) mini-elute column and elution with EB Buffer. The final concentration of the genomic DNA may be about 10 ng/l. To generate extended capture probes 2.5 μl of adaptor ligated DNA, 2.5 μl 10×Taq Gold Buffer, 2 μl 25 mM MgCl2, 2.5 μl 10×dNTPs, 5 μl of a 500 nM mixture of 150 different capture probes in TE buffer, 0.25 μl Perfect Match Enhancer, 0.25 μl AmpliTaq Gold (Applied Biosystems, Foster City, Calif.) and 10 μl of water may be mixed to give a final reaction volume of 25 μl. The reaction may be incubated at 95° C. for 6 min followed by 26 cycles of 95° C. for 30 sec, 68° C. for 2.5 min (decreasing 0.5° C. on each subsequent cycle) and 72° C. for 1 min, then to 4° C.

The extended capture probes may be made double stranded by the addition of 0.25 μl of 1 μM T7 primer and incubation at 95° C. for 2 min, 55° C. for 2 min, 72° C. for 6 min, then to 4° C. The reaction may be passed over a G-25 Sephadex column and 5 μl of 10× Exonuclease I Buffer (NEB) and 2 μl of Exonuclease I (NEB) may be added and the reaction was incubated at 37° C. for 60 min, 80° C. for 20 min, then to 4° C. The products may be purified over a Qiagen (Valencia, Calif.) mini-elute column and eluted with 10 μl EB Buffer.

Generic PCR may be done as follows: 65.5 μl water, 10 μl 10×Taq Gold Buffer, 8 μl 25 mM MgCl2, 10 μl 10×dNTPs, 1 μl 1 μM T3 primer, 1 μl 1 μM T7 primer 3 μl DNA, 0.5 μl Perfect Match Enhancer and 1 μl AmpliTaq Gold were mixed in a 100 μl final reaction volume and incubated at 95° C. for 8 min, 40 cycles of 95° C. for 30 sec, 55° C. for 1 min, and 72° C. for 1 min, then 72° C. for 6 min followed and finally to 4° C.

An aliquot of the reaction may be analyzed on a 2% agarose gel. The products may then be digested with the Type IIs restriction enzyme, BbvI. The digest may be divided into two aliquots. One aliquot is extended in the presence of biotin ddGTP and the other in the presence of biotin ddATP. The extension products from each aliquot may then be hybridized to an array of tag probes under standard conditions and hybridization patterns may be analyzed.

### Example 2

### Analysis of 5-methyl C Using Methylation Sensitive Restriction Enzymes

Digestion: Set up three reactions. In each reaction digest 300 ng human genomic in a 20 μl reaction in 1×NEB buffer 2 with 1×BSA and 1 U/μl Xba1 (NEB). Incubate the reactions at 37° C. overnight or for 16 hours. Heat inactivate the enzyme at 70° C. for 20 minutes.

Ligation: Mix the 20 μl digested DNA with 1.25 μl of 5 μM adaptor, 2.5 μl 10× ligation buffer and 1.25 μl 400 U/μl ligase. The final concentrations are 12 ng/μl DNA, 0.25 μM adaptor, 1× buffer and 20 U/l ligase. Incubate at 16° C. overnight. Heat inactivate enzyme at 70° C. for 20 minutes. Sample may be stored at −20° C. Digest one of the reactions with MspI and a second with HpaII.

Amplification: Mix the ligation reactions in three separate 1000 ul PCR reactions. Final concentrations of reagents may be as follows: 1×PCR buffer, 250 μM dNTPs, 2.5 mM MgCl₂, 0.5 μM primer, 0.3 ng/μl ligated DNA, and 0.1 U/μl Taq Gold. Each reaction may be divided into 10 tubes of 100 μl each prior to PCR.

Reaction cycles may be as follows: 95° C. for 10 minutes; 20 cycles of 95° for 20 seconds, 58° C. for 15 seconds and 72° C. for 15 seconds; and 25 cycles of 95° C. for 20 seconds, 55° C. for 15 seconds, and 72° C. for 15 seconds followed by an incubation at 72° C. for 7 minutes and then incubation at 4° C. indefinitely. Following amplification 3 μl of the sample may be run on a 2% TBE minigel at 100V for 1 hour.

Fragmentation and Labeling: PCR reactions may be cleaned and concentrated using a Qiagen PCR clean up kit according to the manufacturer's instructions. Eluates may be combined to obtain a sample with approximately 20 μg DNA, approximately 250-300 μl of the PCR reaction may be used. The 20 μg product should be in a volume of 43 μl, if necessary vacuum concentration may be required. The DNA in 43 μl may be combined with 5 μl 10×NEB buffer 4, and 2 μl 0.09 U/μl DNase and incubated at 37° C. for 30 min, 95° C. for 10 minutes then to 4° C. DNA may be labeled with TdT under standard conditions.

Hybridization: Each reaction should be hybridized to a separate array. Standard procedures may be used for hybridization, washing, scanning and data analysis. Hybridization may be to an array designed to detect the presence or absence of a collection of human XbaI fragments of 400 to 1,000 base pairs such as the arrays described in U.S. patent application Ser. No. 10/681,773.

### CONCLUSION

From the foregoing it can be seen that the present invention provides a flexible and scalable method for analyzing methlyation in complex samples of DNA, such as genomic DNA. Generally, the invention provides methods for highly multiplexed locus specific amplification of nucleic acids that preserves information about the methylation status of cytosines in the starting sample and determination of methylation status. From experiment design to isolation of desired fragments and hybridization to an appropriate array, the above invention provides for fast, efficient and inexpensive methods of complex nucleic acid analysis.

All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

1. A method for determining if a cytosine in a target sequence in a nucleic acid sample is methylated comprising:
fragmenting the nucleic acid sample to generate fragments;
treating the sample with an agent that modifies unmethylated cytosines but does not modify methylated cytosines;
ligating an adaptor to the fragments, said adaptor comprising a first common sequence;
hybridizing a capture probe to the target sequence wherein the capture probe comprises a second common sequence, a tag sequence, a recognition sequence for a type IIs restriction enzyme and a region that is complementary to a region of the target sequence 3′ of the cytosine;
extending the capture probe to generate an extended capture probe;
amplifying the extended capture probe with first and second common sequence primers to generate double stranded extended capture probes;
digesting the amplified product with a Type IIS restriction enzyme to generate restriction fragments;
extending the restriction fragments in the presence of at least one labeled ddNTP;
hybridizing the restriction fragments to an array of oligonucleotides comprising a probe that is complementary to the tag sequence;
analyzing the hybridization pattern to determine the identity of labeled ddNTPs incorporated into the restriction fragments; and
determining the methylation status of the cytosine from the identity of labeled ddNTPs incorporated.

2-39. (canceled)


SEQUENCE LISTING (28 sequences)

SEQ ID NO: 1 - DNA, 36 bp, Artificial
  Synthetic sequence.
cttttttgaggcatgttcgttttcacttaagaggtt

SEQ ID NO: 2 - DNA, 36 bp, Artificial
  Synthetic sequence.
uttttttgagguatgttcgttttuauttaagaggtt

SEQ ID NO: 3 - DNA, 36 bp, Artificial
  Synthetic sequence.
uttttttgagguatgttugttttuauttaagaggtt

SEQ ID NO: 4 - DNA, 29 bp, Artificial
  Synthetic sequence.
caaaaatacgacgtccaataatcttagaa

SEQ ID NO: 5 - DNA, 36 bp, Artificial
  Synthetic sequence.
uttttttgagguatgttygttttuauttaagaggtt

SEQ ID NO: 6 - DNA, 42 bp, Artificial
  Synthetic sequence.
aagattctaataacctataaaaacraacatacctcaaaaaaa

SEQ ID NO: 7 - DNA, 36 bp, Artificial; identical to SEQ ID NO: 5
  Synthetic Sequence.

SEQ ID NO: 8 - DNA, 48 bp, Artificial
  Synthetic sequence.
aagattctaataacctcgcagcataaaaacraacatacctcaaaaaaa

SEQ ID NO: 9 - DNA, 48 bp, Artificial
  Synthetic sequence.
tttttttgaggtatgttygttttcacgctgcgaggttattagaatctt

SEQ ID NO: 10 - DNA, 63 bp, Artificial
  Synthetic sequence.
gggagacgttcctaaagctgagtctgaagattctaataacctcgcagcataaaaacrgnnnnn

SEQ ID NO: 11 - DNA, 63 bp, Artificial
  Synthetic sequence.
nnnnngygttttcacgctgcgaggttattagaatcttcagactcagctttaggaacgtctccc

SEQ ID NO: 12 - DNA, 56 bp, Artificial
  Synthetic sequence.
gggagacgttcctaaagctgagtctgaagattctaataacctcgcagcataaaaac

SEQ ID NO: 13 - DNA, 60 bp, Artificial
  Synthetic sequence.
nnnygtttttatgctgcgaggttattagaatcttcagactcagctttaggaacgtctccc

SEQ ID NO: 14 - DNA, 57 bp, Artificial
  Synthetic sequence.
gggagacgttcctaaagctgagtctgaagattctaataacctcgcagcgtgaaaacy

SEQ ID NO: 15 - DNA, 60 bp, Artificial
  Synthetic sequence.
nnnygttttcacgctgcgaggttattagaatcttcagactcagctttaggaacgtctccc

SEQ ID NO: 16 - DNA, 30 bp, Artificial
  Synthetic sequence.
tagccatcggtacgtactcaatgatcagct

SEQ ID NO: 17 - DNA, 30 bp, Artificial
  Synthetic sequence.
taguuatuggtaugtautuaatgatuagut

SEQ ID NO: 18 - DNA, 30 bp, Artificial
  Synthetic sequence.
taguuatcggtacgtautuaatgatuagut

SEQ ID NO: 19 - DNA, 25 bp, Artificial
  Synthetic sequence.
atcattaaatacataccaataacta

SEQ ID NO: 20 - DNA, 25 bp, Artificial
  Synthetic sequence.
atcattaaatacttaccaataacta

SEQ ID NO: 21 - DNA, 25 bp, Artificial
  Synthetic sequence.
atcattaaatacgtaccgataacta

SEQ ID NO: 22 - DNA, 25 bp, Artificial
  Synthetic sequence.
atcattaaatacctaccgataacta

SEQ ID NO: 23 - DNA, 25 bp, Artificial
  Synthetic sequence.
actaataattaaatacataccaata

SEQ ID NO: 24 - DNA, 25 bp, Artificial
  Synthetic sequence.
actaataattaattacttaccaata

SEQ ID NO: 25 - DNA, 25 bp, Artificial
  Synthetic sequence.
actaataattaaatacgtaccgata

SEQ ID NO: 26 - DNA, 25 bp, Artificial
  Synthetic sequence.
actaataattaattacctaccgata

SEQ ID NO: 27 - DNA, 11 bp, Artificial
  Synthetic sequence.
ctcttcnnnnn

SEQ ID NO: 28 - DNA, 11 bp, Artificial
  Synthetic sequence.
nnnnngaagag